(19) United States
(12) Patent Application Publication
(10) Pub. No.: US 2023/0080426 A1
(43) Pub. Date: Mar. 16, 2023

(54) ONE TIME USE FLUID METERING DEVICE

(71) Applicant: RXACTUATOR, INC., Tucson, AZ (US)

(72) Inventor: MARK BANISTER, TUCSON, AZ (US)

(21) Appl. No.: 17/904,274

(22) PCT Filed: Feb. 15, 2021
(86) PCT No.: PCT/US2021/018130

§ 371 Date: Aug. 15, 2022

Related U.S. Application Data
(60) Provisional application No. 62/976,966, filed on Feb. 14, 2020.
(60) Provisional application No. 62/988,144, filed on Mar. 11, 2020.

(51) Int. Cl. A61M 5/142; G01F 11/08; A61M 39/10; A61M 5/158

(52) U.S. Cl. CPC A61M 5/14244; A61M 5/158; A61M 39/10; G01F 11/082

(57) ABSTRACT

A wearable fluid metering device for delivering medication subcutaneously, intramuscularly or intravenously to a patient, includes a polymer actuator pump. The polymer actuator pump is flexible so it will conform to shape and yet can be used to rapidly displace a volume within a confined area. The actuator is much faster than prior art polymer actuator designs and can be actuated in multiple ways including introduction and hydration of the polymer matrix by a solution or the actuator can be actuated via pH change while it is in solution. The pH change can be made via electrical current such as that used in electro chemistry or the pH change can be made via a chemical change introduced to the actuator hydrating solution.

Controlled delivery of fluids, such as drugs, in the medical and veterinary fields is accomplished by a variety of methods. One method of controlled prolonged delivery of beneficial agents involves the use of osmotic delivery systems. These devices can be external devices or implanted to release beneficial agents in a controlled manner over a pre-selected time or administration period. In general, osmotic delivery systems operate by absorbing fluid from the outside environment and releasing corresponding amounts of the beneficial agent. These devices are somewhat limited in use and practicality due to inaccuracies or delays in the time it takes for the device to absorb a fluid and start expelling fluid flow out of the device. This delay is caused by air pockets or voids that remain and get compressed during the start of the pump process Delayed start-up of beneficial fluids delivery is a significant problem in osmotic delivery systems.

This problem is solved by using a flexible actuator material that can be compressed either when pump is assembled or when filled with fluid or medication to be released later, creating a preloaded pressure condition that negates any dead airspace compression issues, that can delay the time it takes to start expelling the beneficial fluids. Because the actuator is already under pressure it starts delivering fluids as soon as the fluids are released from any restrictions on the fluid tubing, as the flexible actuator material relaxes outwardly when released. This allows time for the actuator material to start absorbing the hydrating fluids.

Another problem with osmotic pumps is that osmotic pressure often is too low to deliver many of the new more viscous pharmaceutical agents and materials. Methods and materials are described that overcome the osmotic pressure issues with the introduction of protonated molecular repulsive forces, that occur upon hydration of the flexible actuator material and can produce pressures in excess of 50 ponds per square inch per gram of actuator material easily delivering most fluids.

Another problem that is solved by a flexible actuator material is that the device can now be made itself of flexible materials as long as these materials are, less flexible, or stiffer than the actuator material, the more flexible a device is the more desirable it is for comfort of the patient, animal or package configuration.

The foregoing discussion of the prior art derives primarily from my prior U.S. Pat. No. 9,995,295 in which I provide a device for metering a fluid comprising a walled fluid chamber with at least one fluid inlet or fluid outlet port. The device has at least one chamber wall and a movable separator that is in contact with and retains the fluid in the chamber. A porous media substrate and a wicking material, and an actuator formed of a flexible polymer material in contact with the porous media substrate and with the moveable separator are contained within the device. An actuator hydrating solution reservoir within the device is bounded by said at least one chamber wall and includes a hydrating solution inlet port in fluid contact with the porous media substrate. A fluid gate is located between the actuator hydrating solution reservoir and the polymer actuator, effectively keeping the actuator dry. The actuator is adapted to move in a direction and apply pressure to the separator in contact with the fluid in the fluid chamber, thereby dispensing fluid from the fluid chamber, wherein the actuator is formed of a material having varying layers of density and porosity or varying degrees of reacted and unreacted molecular sites.

The polymer gels create volume and pressure changes upon hydration. However these polymer gels and actuator designs are built on polymer materials that are either cast into a geometric shape or ground up to produce a granular powder, which is bound and shaped into an actuator package that fits into a rigid container. The binder, which is needed to add structural strength and form to the powder slows down diffusion and decreases the overall swelling size as the polymer powder matrix is bound more tightly together and becomes more difficult to physically swell.

The present disclosure provides an improved device for metering fluids. More particularly, the present invention disclosure provides fluid metering device or pump that can deliver a pre-determined volume of fluid at a pre-determined pressure at a pre-determined rate of delivery. The device can be pre filled with the fluid during the manufacturing process or the device can be manufactured empty to be filled at a later time. The device can also meter multiple fluids in separate reservoirs at the same time and mix the fluids, if needed. The invention has particular utility as a wearable device for metering drugs through the skin of humans and other animals, and will be described in connection with such utility, although other utilities are contemplated.

The present disclosure in broad aspect comprises a wearable device for metering fluids comprising one or more fluid chambers each with at least one inlet, at least one outlet port, and at least one sidewall, wherein an interior of the sidewall is in contact with and retains or separates fluid in the fluid chamber from other components of the device. The device includes one or more actuator assemblies formed of one or more polymer actuator materials, with one or more polymer actuator material holders in contact with the polymer actuator material. A wicking material is provided in contact with the polymer actuator material. The device also includes a flexible outer shell that is sealed to retain a polymer actuator hydrating solution, and including a hydrating solution inlet port configured to provide a fluid path for the polymer actuator hydrating solution. A one way valve is provided configured to allow hydrating fluid into the actuator assembly but not out. The device also includes a connector configured to allow tubing to be attached prior to hydration of the polymer actuator material, and removed once the hydrating solution has been delivered into the actuator assembly. One or more platens are located between the actuator assembly outer shell and the exterior of the metered fluid chamber. The actuator hydrating solution is held within an actuator hydrating solution reservoir having at least one sidewall, and one or more inlet or outlet ports, in fluid communication via a removable tube or fluid path connecting the actuator hydrating solution to the polymer actuator assembly. A fluid gate is provided configured to open or close the fluid path located between the hydrating solution reservoir and the actuator assembly, effectively keeping the actuator material dry until the gate is opened. The device also includes a rigid or semi rigid external shell configured to encase the actuator assembly, platen and fluid chamber and hold all components other than the hydration solution reservoir which is removable, whereby the polymer actuator assembly in contact with the platen, once hydrated is configured to expand in a desired direction and apply pressure to the platen which in turn in contact with the fluid chamber, applies pressure to the fluid chamber whereby to dispense fluid from fluid chamber. Completing the device is a woven or nonwoven fabric adhered to the exterior case, or tabs extending from the exterior of the case, configured to facilitate attachment of the device to the skin of the wearer using a tape or adhesive, or sutures or staples.

In one aspect of the invention, the fluid chamber and/or the polymer actuator assembly is removable.

In another aspect of the invention the fluid chamber and/or the actuator housing is formed of a polyvinyl, plastic, metal, glass, ceramic, carbon or a combination thereof.

In still another aspect of the invention the fluid chamber inlet port is capped or sealed by a pierceable septum.

In another aspect of the invention the movable separator is a rubber plunger.

In another aspect of the invention the polymer actuator material is formed of a hydrophilic material, or is a combination of a hydrophilic and a hydrophobic material.

In still yet another aspect of the invention the fluid gate is removable by piercing, dissolving, tearing, pushing, pinching the tubing connector or by pulling the gate away from hydrating solution path to allow hydration of the polymer actuator.

In a further aspect of the invention the fluid gate comprises a membrane material that can be burst, pierced or dissolved.

In another aspect of the invention the fluid gate comprises a mechanical valve.

In yet another aspect of the invention the fluid in the fluid chamber comprises a medication or therapeutic material.

In another aspect of the invention the fluid chamber is configured for filling in the device.

In still another aspect of the invention the fluid chamber is configured for filling via an injection of fluid into the septum and fluid in the fluid chamber is released into outlet tubing that is in contact with a wearer via a subcutaneous needle, an intramuscular needle, an intravenous needle, a catheter or a luer connection that allows metering of released fluid to a desired wearer contact point.

In yet another aspect of the invention the device includes a subcutaneous needle, an intramuscular needle, an intravenous needle, a catheter or a luer connection configured to allow metering of released fluid directly to a desired contact point of a wearer.

In another aspect of the invention the actuator material has varying layers of density and porosity.

In another aspect of the invention the actuator material has both reacted and unreacted molecular sites, preferably varying degrees of reacted and unreacted molecular sites.

In yet another aspect of the invention the actuator hydrating solution comprises a combination of actuator materials having a ratio of reacted to unreacted molecular sites selected to determine the speed and pressure generation of the actuator material.

In still yet another aspect of the invention protonation of reactive molecular sites within the actuator material, by interaction with the actuator hydrating solution or chemical byproduct of that interaction, determines a speed and pressure generation of the actuator material.

In another aspect of the invention the actuator materials are selected by density or porosity to determine a speed and pressure generation of the actuator material.

In yet another aspect of the invention the actuator material surface in fluid contact with the actuator hydrating solution material is configured to determine a speed and pressure generation of the actuator.

In still yet another aspect of the invention the actuator hydrating solution pH or chemical makeup is selected to determine a speed and pressure generation of the actuator material.

In another aspect of the invention the device rigid or semi rigid exterior case has one or more windows or visual ports configured to allow visualization of a metered fluid volume indicator. In another preferred embodiment the platen is the metered fluid volume indicator.

In another aspect of the invention the device rigid or semi-rigid exterior case has a door or hatch configured to cover the two fill ports and latch closed when the fluid chamber is filled and the actuator activated, so that the device cannot be reopened during the use of the metering device.

In yet another aspect of the invention the device rigid or semi-rigid exterior case has an interior pocket configured to shield and protect the outlet tubing where it exits the exterior case, and located in the vicinity of the interior pocket the outlet tubing has a flow restrictor or anti siphon valve in the fluid path to stop un-authorized removal or theft of medications from the device while it is operating.

In still yet another aspect of the invention the actuator hydrating solution container comprises a syringe or other containment device which is configured to be removably clipped to the external shell of the device, and removed once the metering device is activated by hydrating the actuator assembly.

In another aspect of the invention the actuator hydrating solution container is contained within the external shell of the device, and the external shell is hinged like a clam shell which is configured to release the actuator hydrating solution into the actuator assembly when the external shell is shut. In such aspect shutting the clam shell to release the hydrating solution, also activates the metering device, and wherein activation of the clam shell triggers a spring loaded subcutaneous needle and canula to project through a side of the exterior case into a wearer’s skin, wherein the needle is configured to retract back into the exterior case in the same or subsequent action, leaving the canula in a subcutaneous layer below the skin of the wearer and in fluid connectivity to the fluid chamber.

In yet another aspect of the invention, one or more types of medication, gene therapies, proteins are metered to a patient via the best route to deliver the particular medications at a desired site on or in the patient.

The present disclosure in another aspect provides improvements to actuator polymer materials. More particularly, I have discovered that significant actuator performance improvement may be achieved by changing the physical shape and form of the actuator gel by deconstructing the polymer gel, into small particles, and then reassembling the particles into shaped structures having increased surface area. In one preferred embodiment, the polymer gel is ground into small particles. Grinding the polymer gel into small particles, e.g., ≤ 1 µm, preferably ≤ 1,000 µm, more preferably in a combination and variety of sizes increases the gel surface area and decreases the distance the hydrating solution has to travel during diffusion into the polymer matrix of the gel. Diffusion is a limiting factor on the speed of these actuators as they can only swell as fast as the hydrating solution can diffuse through the polymer matrix.

Polymer actuators are limited due to design constraints such as how to physically hold a slurry made from the ground polymer. The size of a cast or molded gel actuator that can be made is limited due to the time it takes for diffusion to occur through the polymer, i.e., the outside of the cast shape swells significantly during hydration and if the cast shape is too large the outside material will shear off as it swells before the inside material starts hydrating.

To overcome these issues an actuator has been developed in which the physical form of the actuator gel is changed to as to create an extremely high surface area and an extremely short distance to reach full hydration of the polymer matrix. More particularly, the liquid polymer gel is cured, partially hydrated, then deconstructed into small particles so as to increase the gel surface area. Additionally, the polymer gel is formed into a structure having an increased surface area to volume.

In one preferred embodiment of the disclosure, the polymer gel is cast onto a substrate by wetting a flexible substrate such as a cloth with the polymer gel in liquid form and then squeezing out any excess liquid polymer gel before allowing the polymer gel material to cure or dry on the cloth. The cloth can be made from many materials such as natural cotton or silk for example, or from manmade materials such as polypropylene, polyester, polyether, nylon, spandex etc.. By coating the fibers of a cloth with a very thin micrometer or nanometer thick layer of polymer gel the cloth provides the structural component needed for support without slowing down the hydration of the ultra-thin layer of polymer on the cloth fibers, allowing for very fast wetting and complete hydration of the polymer matrix in mere seconds as compared to minutes for cast actuators formed from ground polymer materials.

This type of actuator design requires a polymer gel that is elastic and flexible when dry, such as a commercially available epoxy polymer smart material made from Huntsman Jeffamine™ polymer reacted with a polyethylene glycol diglycidyl ether and water. The Jeffamine™ polymer materials have very flexible backbones with two or more branches that are terminated with amines that react and crosslink with the active oxygen molecules of the diglycidyl ether. There are many combinations of these type of polymers that can be crosslinked together by various methods that would be obvious to those in the art. Such as adding UV crosslinkers or chemical crosslinkers to the formulations. It is important to note, however, that more common acrylamide polymer gels do not work well as they become hard and brittle when dry and detach from the cloth. Beyond this acrylamide gels can break down into toxic substances over time and with UV exposure. This makes them undesirable in this use. In the present disclosure, the polymer coated cloth or substrate, both in woven and nonwoven forms, limit the expansion of the length and width of the actuator, if the cloth is held in a horizontal plane. The cloth will however expand in thickness as that is the path of least resistance for the polymer coated fibers. By stacking multiple layers of the coated cloth or substrate the layers will swell rapidly in unison and create a sizable vertical displacement or actuation stroke and pressure at a much more rapid speed than previous designs that use cast shapes or finely ground polymer materials.

In one preferred embodiment the actuator assembly consists of an outer flexible shell with one inlet opening and an internal stack of one or more actuator polymer coated cloth pieces cut in any geometric shape. In another preferred embodiment the actuator assembly consists of a roll of actuator polymer wrapped around a flexible metered fluid container that is held a flexible metered fluid container with one or more inlets and outlets, wherein the metered fluid container is wrapped or surrounded with the actuator polymer coated cloth and held within a rigid or semi-rigid outer shell with one or more openings that allow hydration of the actuator material.

In another aspect of the invention the actuator polymer coated cloth layers are connected together in such a way that they stay assembled together and can not move or separate apart once assembled for ease of manufacturing and assembly of various actuator configurations.

In another preferred embodiment the actuator polymer coated cloth layers are heat staked or heat sealed together during assembly in one or more locations, wherein the layers can still swell without restriction in the areas that are not heat staked or heat sealed. In another aspect of the invention the actuator polymer coated cloth layers are sewn together during assembly in one or more locations, wherein the layers can still swell without restriction in the areas that are not sewn together. In another aspect the actuator polymer coated fabric layers may be glued together during assembly in one or more locations, wherein the layers can still swell without restriction in the areas that are not glued together or any combination of theses methods described.

In order to electrically or chemically activate the actuator the actuator polymer coated cloth is simply exposed to and hydrated by a liquid electrolyte and stacked in layers with one or more anodes and one or more cathodes in the same electrolyte solution. The electrodes can be made of common electrode materials such as metals, foils, graphite etc. In a preferred embodiment the cathodes or anodes may be made of very thin materials such as metal foils and incorporated into the stacked layers. While the opposing electrode is in the form of an exposed wire traveling the length of the stack as well as the stacks stroke length but not in contact with the opposing electrode. This ensures that the electrical current will still flow between electrodes as the actuator expands. In another embodiment the opposing electrodes can be positioned at each end of the stack with the electrolyte ensuring electrical current connectivity. In yet another embodiment one or more electrodes are deposited via vapor deposition to the inside wall of an actuator container traveling the length of the container wall and can be connected electrically. In another preferred embodiment the actuator comprises an outer shell that is rigid or a bag in an outer shell that is stiffer or more rigid than the actuator material, and is sealed in such a way that hydrating solution cannot leak out once it is introduced, i.e., similar to how batteries are manufactured, and includes the use of metalized polymer or plastic films that will stop evaporation of electrolytes through the film, as well as heat sealing films around electrodes that stop electrolyte migration along the electrode.

In a preferred embodiment the hydrating solution can be water, or other liquids of different pH ranges can be used to cause the actuator to swell or expand to desired dimension. For electrically controlled and reversable activation the hydrating solution typically is a salt solution for ion transport efficiency and if reversing motion is desired the hydrating solution must be able to reverse back to its original chemical make up when the electricity is reversed changing the charge at the electrode site causing pH change in the solution.

Electrical activation using a power source and a programmable controler changes the pH as the charge is applied to the electrodes in the actuator assembly current will travel through the solution and change the pH of the solution at the electrode region this pH change can be acidic or base dependent of the hydrating solutions chemical makeup and electrode pole. For example, sodium acetate solution will convert to acetic acid at the positive electrode and revert a base solution of sodium acetate at the negative electrode. Many salt solutions will change pH at the electrodes while the salt concentration determines the electrical resistance or conductivity of the solution. These are well known in the art of electrochemistry.

In another embodiment of the disclosure, the actuator material or actuator assembly comprises an outer container or shell that is sealed to prevent evaporation or water vapor loss. A metalized plastic bag material commonly is used within the outer shell. As before, the outer shell is stiffer or more rigid than the actuator material. Inside the container is the stack of polymer gel coated cloth or substrates. Thin metal foil electrodes are alternately stacked along with the polymer gel coated cloth or substrate layers. The thin metal foil electrodes all have a common connecting electrical conductor that exits the outer container. One or more counter electrodes are sealed within a layer of ion separator materials and are located around the exterior of the stacked materials with a common electrical connector that exits the outer container. The container and actuator stack and electrodes are assembled in such a way that there are only two electrical conductors exiting the outer container. The assembly is filled with a salt solution and sealed in such a way that the salt solution cannot leak out. This may be accomplished via heat sealing, ultrasonic welding or other known methods. The hydrated polymer gel actuator material on cloth substrate further swells in the presence of an acid. An acid is created at the positive electrodes and the electrodes are sandwiched in between the actuator material layers. The counter electrode is positioned so that it is separated from the positive electrodes either by distance or with an ion separator material. This provides definitive separation of the electrode pH change regions within the contained actuator material and solution. Positive current via the controler is sent through electrodes in between the layers of the stacked materials and negative current is sent through the counter electrode. This produces acidic conditions that swell the polymer gel substrates in the stack more than it is, in its standing or equalized hydrated state, this can be as much as 100-200% larger. The stack assembly extends vertically due to the swelling of each polymer gel substrate layer of the stack. Reverse the polarity of each electrical conductor and the stack will reverse the swelling and de-swell. If the actuator assembly is run at more than 3.2 volts of electrical input, gas generation from electrolysis can occur with water based salt solutions. The gas generation can be mitigated by using activated carbon coating of the positive electrodes using the methods developed for double layer capacitor electrodes and incorporating them into the stack assembly. The gas generation from electrolysis will be stopped until the electrode is fully charged like a capacitor at that point the electrode can be shorted or discharged and gas generation eliminated again, this can be done repeatedly.

In another preferred embodiment of the disclosure the polymer gel substrate cloth is cut out in the shape of a flattened donut or ring with one or a plurality of through holes and stacked with their through holes lined up, as an actuator for a syringe or syringe like tube with openings at both ends. One end may contain a liquid in direct contact with a rubber plunger, to be delivered, and the actuator is located in the tube end behind the rubber stopper and in contact with the rubber stopper. A rubber plunger is configured to slide and separate the liquid being delivered or stored from the actuator stack. The actuator stack is contained at the back of the tube by a cover that attaches so it can constrain the actuator stack and has hydration fluid port. The hydration fluid port allows one-way transfer of hydrating solution from a hydrating solution storage container which may or may not be attached to the syringe or syringe like tube cylinder. To activate the actuator, hydration solution is introduced to the actuator stack at the back of the cylinder and rubber stopper. The actuator stack quickly absorbs the hydrating solution, expands in a vertical direction and pushes the rubber stopper which in turn pushes out the contents of the syringe or syringe like tube. The back cover of the syringe or syringe like tube may or may not contain an air vent to allow air in or out but not allow hydrating solution out. There are several types of hydrophobic air filters and vents that allow airflow but stop liquid migration through the material such as micropore or sintered porous plastic that work very well for this and are well known in the art.

Further features and advantages of the present disclosure will be seen from the following detailed description, taken in conjunction with the accompanying drawings, wherein

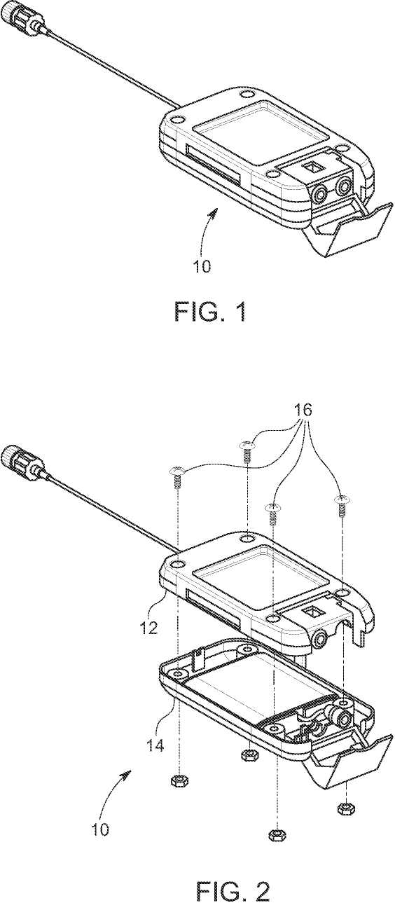

FIG. 1 is a perspective view of a metering device in accordance with one embodiment the present disclosure;

FIG. 2 is an exploded view of the fluid metering device of FIG. 1;

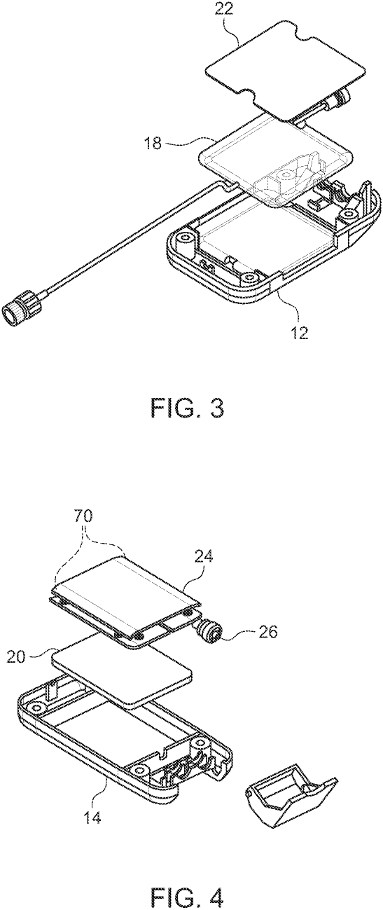

FIGS. 3 and 4 are exploded views showing details of the upper case and lower case, respectively of the fluid metering device of FIG. 1;

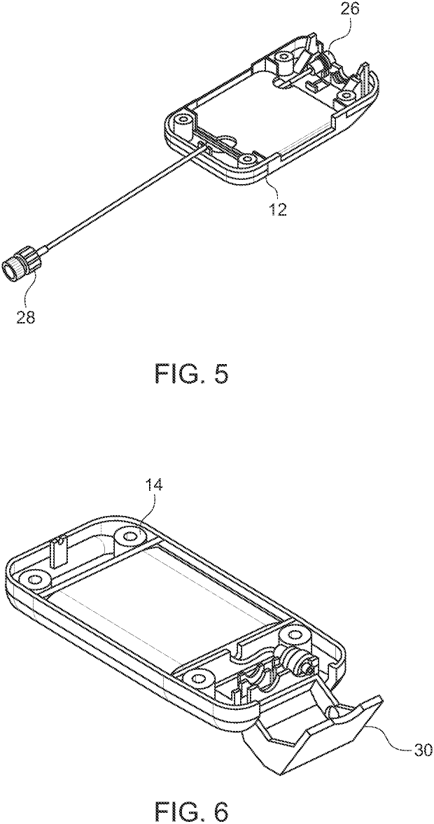

FIGS. 5 and 6 are perspective views showing details of the upper case and lower case, respectively, partially assembled;

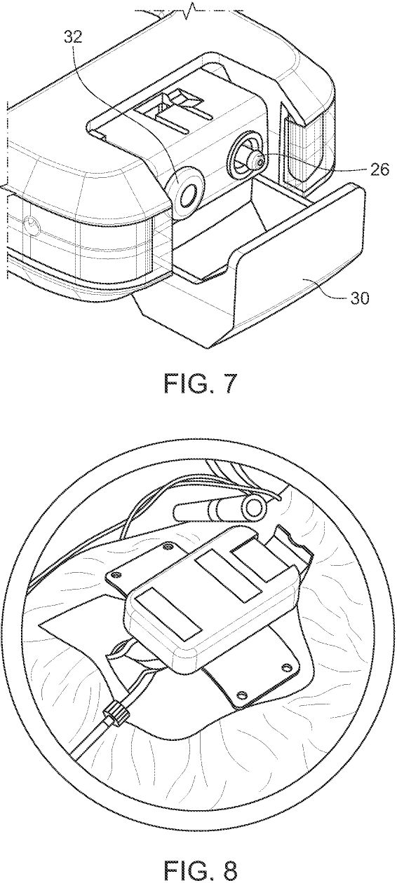

FIG. 7 is an enlarged view showing details of an end view of the metering device of FIG. 1;

FIG. 8 is a perspective view showing the fluid metering device of the present invention taped to the back of a patient;

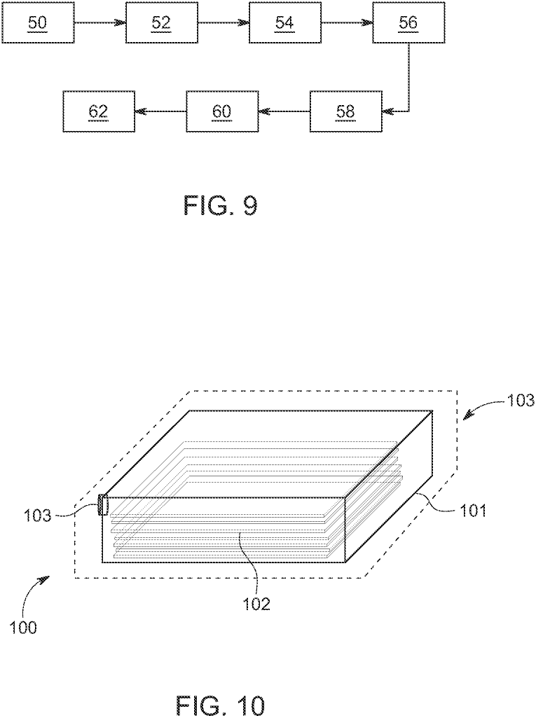

FIG. 9 is a flow diagram showing use of the fluid metering device of the present disclosure;

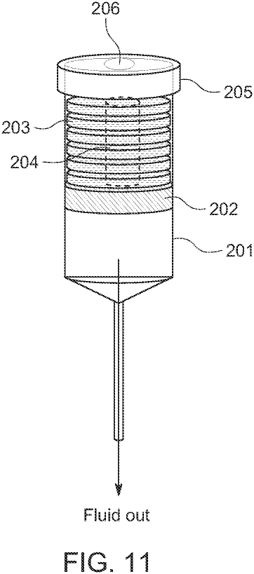

FIG. 10 is a perspective view of a dry stacked actuator assembly made in accordance with a a second embodiment of the present disclosure; and

FIG. 11 is a view, in cross section, of a syringe incorporating actuator assembly made in accordance with the present disclosure.

Referring to FIGS. 1-7, fluid metering device 10 of a first embodiment of the present disclosure comprises a shell including an upper case 12 and a lower case 14 each formed of a rigid or semi-rigid material such as vinyl, plastic, metal, glass, ceramic, carbon or a combination thereof, affixed to one another by screws 16. A window 17 may be provided in the wall of upper case 12 to permit a caretaker to view the drug chamber to allow visualization of a metered fluid volume indicator. Referring in particular to FIGS. 3 and 4, the fluid metering device includes a drug chamber 18 formed of a flexible bladder sandwiched between an upper and lower platens 20, 22. An actuator assembly 24 is disposed over platen 22. Actuator assembly 24 comprises a flexible chamber containing a polymer actuator material formed of a hydrophilic material or a combination of a hydrophilic material and a hydrophobic material such as hydrogel polymer as described in U.S. Application 2014/0221913 or PCT Application WO 2008/07944082, the contents of which are incorporated herein by reference. Drug holding chamber 18 includes an inlet or fill port 26 and an outlet 28 preferably in the form of a luer connector as will be described below.

As will be described below, when water is introduced into the actuator assembly 24 via a fluid gate such as a one way valve or pierceable septum 32, the polymer actuator material swells to a controlled linear rate and creates a high pressure. The fluid gate also may be removable by piercing, dissolving, tearing, pushing, pinching the tubing connector or by pulling the gate away from hydrating solution path to allow hydration of the polymer actuator, or a membrane material that can be burst, pierced or dissolved, or a mechanical valve. High pressure created by the swelling polymer is transferred via platen 22 to the drug holding chamber which then pushes medication out of the metering device at a very linear and controlled rate.

Fill port 26, which typically comprises a tubing connector or the like is protected behind a hinged door 30, which door protects fill port 26 and septum 32, and covers the two fill ports and latch closed when the fluid chamber is filled and the actuator activated, so that the device cannot be reopened during the use of the metering device.

Referring also to FIGS. 8 and 9, use of the above described metering device of the present invention will now be described.

In step 50, the patient’s weight and medication therapy level is determined. Then in step 52, using a simple look-up table a, e.g., 10 mL supply of desired medication at a desired concentration or dilution is prepared. The diluted 10 mL drug is then injected into the drug fill port 26 at step 54 using a hypodermic needle or the like and tubing. The metering device 10 is then fixed to the patient’s skin by suture or stapling, adhesive tape, or wrapping around a limb or torso of the patient in a step 56, and the luer connector 28 is affixed to a subcutaneous needle, an intramuscular needle, an intravenous needle, a catheter or a luer connection that allows metering of released fluid to a desired wearer contact point, that was previously inserted into the patient in a step 58. A measured quantity of water is then supplied via septum to the actuator assembly 24, for example, using a syringe, in step 60, and the door 30 is closed at step 62. The actuator hydrating solution comprises a combination of actuator materials having a ratio of reacted to unreacted molecular sites selected to determine the speed and pressure generation of the actuator material. The actuator hydrating solution pH or chemical makeup is selected to determine a speed and pressure generation of the actuator material. Alternatively, the pump starts automatically and as the polymer actuator swells it puts pressure in the drug holding chamber 18 and delivers drug at a very linear rate.

As will be appreciated, the above described device has several features and advantages. The metering device can be worn by a patient without any external leads, lines or tethers. The metering device provides extremely constant subcutaneous flow. Also, being affixed immediately adjacent to subcutaneous catheter, there is little likelihood of IV line pullout.

Also, not being tied to external lines or tethers, the patients can ambulate without disruption of medication delivery. And, by eliminating the need for towers, expensive pumps, and electrical power, the fluid metering device of the present invention eliminates substantial capital equipment expenditures and associated carrying costs. Also, drug delivery calculations are simplified since capacity of the drug reservoir is standardized. Thus it is a simple dilution calculation to determine dosage. As a result, medication errors may be reduced.

The specific embodiments disclosed and illustrated herein should not be considered as limiting the scope of the disclosure, and numerous variations are possible. By way of example, actuating assembly 24 may include one or more internal wicks, shown in phantom at 70, to even the delivery of the hydrating water into the actuating assembly. Also, the device rigid or semi-rigid exterior case has an interior pocket configured to shield and protect the outlet tubing where it exits the exterior case, and located in the vicinity of the interior pocket the outlet tubing has a flow restrictor or anti siphon valve in the fluid path to stop un-authorized removal or theft of medications from the device while it is operating. Also, the actuator hydrating solution may container comprise a syringe or other containment device which is configured to be removably clipped to the external shell of the device, and removed once the metering device is activated by hydrating the actuator assembly. Still other product features are possible. For example, the actuator hydrating solution container may be contained within the external shell of the device, and the external shell is hinged like a clam shell which is configured to release the actuator hydrating solution into the actuator assembly when the external shell is shut, or wherein shutting the clam shell to release the hydrating solution, also activates the metering device, and wherein activation of the clam shell triggers a spring loaded subcutaneous needle and canula to project through a side of the exterior case into a wearer’s skin, wherein the needle is configured to retract back into the exterior case in the same action, leaving the canula in a subcutaneous layer below the skin of the wearer and in fluid connectivity to the fluid chamber.

Still other variations are possible. For example, referring to FIG. 10, in yet another embodiment an actuator assembly 100 comprises a flexible container 101 formed of a poly bag film with a stack of flexible polymer gel loaded substrates 102 and a fill port where polymer gel hydrating liquid can be injected is provided. A rigid container shown in phantom at 103 surrounds flexible container 101.

Referring to FIG. 11, in yet another embodiment the actuator assembly is held within a syringe tube 201 having a moveable rubber plunger 202. The actuator assembly is formed of donut shaped actuator substrates 203 stacked with holes in the centers 204. A tube end cap 205 constricts the actuator stack and includes a fill port 206 for introducing hydrating polymer to the actuator stack. The syringe tube is made of glass or a rigid plastic that is compatible with the liquid that will be dispensed such as a medication or drug or chemical. Alternatively, actuator substrates 203 may include a plurality of aligned through holes to carry the activating liquid.

The foregoing description of the disclosure is not meant to limit it in any way as there can be many other variants of the described disclosure such as multiple cylinders and actuators that can be activated together or separately at predetermined times using a power source such as a battery, electrical controller, electrical circuitry and program or by simple mechanical means of timed release by a clock and one or more mechanical valves. Additionally, envisioned are actuators for devices that are shaped to fit a contour such as on a human body, or other geometric shapes. Also, if desired, the actuator assembly, and/or the metered fluid chamber, and/or the actuator hydrating solution reservoir, and/or the needle, etc. connection may be removable and replaceable so that the outer shell and electronics may be reused, while the removable parts are in a single assembly or cartridge.

The subject matter of the disclosure includes all novel and nonobvious combinations and subcombinations of the various elements, features, functions, and/or properties disclosed herein.

1-15. (canceled)

16. A wearable device for metering fluids comprising;
a rigid or semi rigid outer shell;
at least one metered fluid chamber having at least one inlet, at least one outlet, and at least one sidewall, wherein an interior of the sidewall is in contact with and retains or separates fluid in the fluid chamber from other components of the device, said device comprising
at least one actuator assembly comprising a flexible outer shell containing at least one polymer actuator material and a wicking material in contact with the polymer actuator material, said actuator assembly having an inlet configured to provide a fluid path for a polymer actuator hydrating solution, and including a one way valve configured to allow flow of hydrating fluid into but not out of the actuator assembly;
a connector configured to allow introduction of a hydrating solution into the actuator assembly;
at least one platen located between an exterior wall of the actuator assembly outer shell and an exterior wall the metered fluid chamber;
an actuator hydrating solution reservoir having one or more inlets or outlets, in fluid communication via a fluid path for delivering the actuator hydrating solution to the polymer actuator assembly; and
a fluid gate configured to open or close the fluid path between the hydrating solution reservoir and the actuator assembly, effectively keeping the actuator material dry until the gate is opened;
wherein the rigid or semi rigid external shell is configured to encase the actuator assembly, the platen and the metered fluid chamber and hold all components other than the hydration solution reservoir, whereby the polymer actuator assembly in contact with the at least one platen, once hydrated is configured to expand and apply pressure to the at least one platen which in turn in contact with the fluid chamber, applies pressure to the fluid chamber whereby to force fluid from fluid chamber.

17. The device of claim 16 where the fluid chamber and/or the polymer actuator assembly and/or the hydration solution reservoir is/are removable.

18. The device of claim 16, characterized by one or more of the following features:
(a) wherein the fluid chamber inlet is capped or sealed by a pierceable septum;
(b) wherein the polymer actuator material comprises a hydrophilic material, or a combination of a hydrophilic and a hydrophobic material;
(c) wherein the fluid gate is configured to open or close by piercing, dissolving, tearing, pushing, bursting, pinching the connector, or by pulling the fluid gate out of the way of the hydrating solution path to allow path of hydration fluid to the polymer actuator;
(d) wherein the fluid gate comprises a mechanical valve; and
(e) further comprising tubing removably attached to the device for introducing the hydrating solution into the device.

19. The device of claim 16, characterized by one or both of the following features:
(a) wherein the metered fluid chamber is configured for filling in the device;
(b) wherein the metered fluid chamber is configured for filling via an injection of fluid into the septum and fluid in the metered fluid chamber is released through to a desired wearer contact point; and
(c) said device optionally further comprising a subcutaneous needle, an intramuscular needle, an intravenous needle, a catheter or a luer connection configured to allow metering of released fluid directly to a desired contact point of a wearer.

20. The device of claim 16 characterized by one or more of the following features:
(a) wherein the actuator material has varying layers of density and porosity;
(b) wherein the actuator material has both reacted and unreacted molecular sites, preferably varying degrees of reacted and unreacted molecular sites;
(c) wherein the actuator hydrating solution comprises a combination of actuator materials having a ratio of reacted to unreacted molecular sites selected to determine a speed and pressure generation of the actuator material, and preferably wherein protonation of reactive molecular sites within the actuator material, by interaction with the actuator hydrating solution or chemical byproduct of that interaction, determines the speed and pressure generation of the actuator material;
(d) wherein the actuator materials are selected by density or porosity to determine a speed and pressure generation of the actuator material;
(e) wherein the actuator material surface in fluid contact with the actuator hydrating solution material is configured to determine a speed and pressure generation of the actuator; and
(f) wherein the actuator hydrating solution pH or chemical makeup is selected to determine a speed and pressure generation of the actuator material.

21. The device of claim 16, characterized by one or more of the following features:
(a) the device rigid or semi rigid exterior case has one or more windows configured to allow visualization of a metered fluid volume indicator;
(b) wherein the device rigid or semi-rigid exterior case has a door or hatch configured to cover the two fill ports and latch closed when the fluid chamber is filled and the actuator activated, so that the device cannot be reopened during the use; and
(c) wherein the device rigid or semi-rigid exterior case has an interior pocket configured to shield and protect outlet tubing running from the exterior case, and wherein the outlet tubing has a flow restrictor or anti siphon valve in the fluid path to stop un-authorized removal or theft of medications from the device;
(d) wherein the rigid or semi rigid outer shell comprises a syringe.

22. The device of claim 16, wherein the actuator hydrating solution container is contained within the outer shell of the device, and the outer shell is hinged like a clam shell and is configured to release the actuator hydrating solution into the actuator assembly when the outer shell is shut, and wherein the device optionally is configured so that shutting the clam shell activates the metering device, and wherein activation triggers a spring loaded subcutaneous needle and canula to project through a side of the exterior case into a wearer’s skin, wherein the needle is configured to retract back into the exterior case in the same action, leaving the canula in a subcutaneous layer below the skin of the wearer and in fluid connectivity to the fluid chamber.

23. The device in claim 22, wherein the device is configured so the action of closing the device additionally pierces a septum or housing of a prefilled fluid container thereby making fluid connectivity between prefilled fluid container and subcutaneous fluid exit point.

24. The device of claim 16, wherein the actuator is electrically activatable and the actuator assembly includes electrodes configured to eliminate electrolysis gas production at the electrodes via double layer capacitor, and wherein said device also contains a power source and electrical controller connected to the actuator.

25. The device of claim 16, wherein the actuator is electrically activatable and the actuator assembly includes electrodes configured to eliminate electrolysis gas production at the electrodes via carbon double layer capacitor, and wherein said device also contains a power source and electrical controller connected to the actuator and said controller is in wireless communication with a sensor and or hand held device wireless device.

26. The device of claim 16, wherein the actuator assembly comprises a flexible container containing a stack of flexible polymer gel loaded substrates, wherein the stack of flexible polymer gel loaded substrates are donut shaped having central holes which are aligned at least in part in said stack, or the stack of flexible polymer gel loaded substrates is formed by wrapping the flexible polymer gel loaded substrates around the metered fluid container, or the stack of flexible polymer gel loaded substrates is formed by folding the flexible polymer gel substrate, or the stack of flexible polymer gel loaded substrates is heat staked or heat sealed or sewn or glued together in one or more locations.

27. The device of claim 16, wherein the fluid in the fluid chamber comprises a medication, a therapeutic material, a gene therapy or a protein, and optionally wherein said device is configured to deliver said medication, therapeutic material, gene therapy or protein to a predetermined site on or in a patient.

28. An actuator assembly comprising a water tight outer shell that is configured to be reversibly extended in at least one direction, a stack or roll of flexible polymer gel loaded substrate, an electrolyte, at least one anode and at least one cathode each extending through the outer shell, and wherein at least one of said anode or cathode is coated with activated carbon and is ionically separated from an opposing cathode or anode as the case may be via an ion separator material.

29. An actuator assembly comprising an outer shell with an inlet port or opening that is in fluid connectivity with a one way valve configured to allow liquid in but not out, wherein the outer shell is configured to expand or contract in at least on direction, said actuator assembly further including a stack of two or more flexible polymer gel loaded substrates within the outer shell.

30. The actuator assembly of claim 29, wherein the flexible polymer gel loaded substrates have holes which are aligned at least in part within the outer shell, and wherein the flexible polymer gel loaded substrates preferably are donut shaped.
